(19) United States
(12) Patent Application Publication
(10) Pub. No.: US 2023/0008024 A1
(43) Pub. Date: Jan. 12, 2023

(54) ANTIBACTERIAL CARBOHYDRATE VACCINE

(71) Applicant: UNIVERSITY OF MONTANA, Missoula, MT (US)

(72) Inventors: Laura K. JENNINGS, Missoula, MT (US); Patrick R. SECOR, Missoula, MT (US); Jay EVANS, Missoula, MT (US)

(21) Appl. No.: 17/781,187

(22) PCT Filed: Dec. 3, 2020
(86) PCT No.: PCT/US20/63086

§ 371 Date: May 31, 2022

Related U.S. Application Data
(60) Provisional application No. 62/943,589, filed on Dec. 4, 2019.

(51) Int. Cl. A61K 39/104; C08B 37/00; A61K 39/385; A61P 31/04

(52) U.S. Cl. CPC A61K 39/104; C08B 37/0063; A61K 39/385; A61P 31/04; A61K 2039/6037

(57) ABSTRACT

The present disclosure provides compositions comprising an isolated polysaccharide comprising β-1,4 linked galactosamine and glucosamine monomers, wherein the amino groups of each of the galactosamine and glucosamine are partially substituted with acetate. The disclosure further provides vaccine, methods of use, and methods of producing the isolated polysaccharide.

### CROSS REFERENCE TO RELATED APPLICATIONS

This application claims the benefit of U.S. Provisional Application No. 62/943,589, filed on Dec. 4, 2019, the contents of which is incorporated herein by reference.

### FIELD

The present disclosure provides compositions, vaccines and methods of use for treating and preventing Pseudomonas aeruginosa infections.

### BACKGROUND

P. aeruginosa is an opportunistic pathogen that infects millions of people annually causing life-threatening infections especially in those inflicted with chronic wounds and individuals with the genetic disorder cystic fibrosis. Due to the increasing prevalence of antibiotic resistant strains, the World Health Organization recently designated P. aeruginosa as a priority pathogen of the greatest risk to human health. A vaccine to treat or prevent P. aeruginosa infections would be ideal to circumvent these problems. However, there is currently no commercially available vaccine that is effective against P. aeruginosa.

One major challenge in vaccine design is to identify a suitable target antigen that is highly immunogenic and sufficiently conserved to be broadly protective. For example, vaccines targeting P aeruginosa lipopolysaccharides, the major component of the outer membrane, fail to generate broad protection against the diverse isolates of P. aeruginosa that cause human infection.

### SUMMARY

Disclosed herein are compositions comprising a polysaccharide comprising of β-1,4 linked galactosamine and glucosamine, wherein the amino groups of each of the galactosamine and glucosamine are partially substituted with acetate. In some embodiments, the polysaccharide is isolated.

Also disclosed herein are vaccines, methods of use, and methods of producing the isolated polysaccharide.

Other aspects and embodiments of the disclosure will be apparent in light of the following detailed description and accompanying figures.

### BRIEF DESCRIPTION OF THE DRAWINGS

FIG. 1 is a graph of the average molecular weight of secreted Pel at 0.5 kDa determined by comparison of Pel, detected with α-Pel immunoblot in size-exclusion fractions, to dextran and cellobiose standards, detected with a colorimetric assay for neutral sugars.

FIG. 2 is graphs of glycosyl composition and linkage analyses of P_{BAD}pel and Δpel culture supernatant indicating that Pel is composed of 1→4 glycosidic linkages of GalNAc and GlcNAc.

FIG. 3 is a graph showing Pel is cationic and partially deacetylated. Supernatant from P_{BAD}pel, but not from Δpel, bound a strong-cation exchange column at pH 5.5 and eluted at 1.25 M NaCl. Fractions from the cation exchange column were probed with Pel antiserum (Top). Load indicates immunoblot of samples loaded to the column. Results shown are from a step gradient (Bottom).

FIG. 4 is a schematic of the vaccination protocol to characterize cell-associated and humoral immunity. Mice were vaccinated with purified Pel or Pel-carrier conjugate and various adjuvants. Immunity was evaluated by measuring anti-Pel antibodies and cytokines.

FIG. 5 is a schematic of a murine pneumonia challenge model. The efficacy of the vaccine will be evaluated by measuring P. aeruginosa (Pa) dissemination to distal tissues in vaccinated and mock-vaccinated mice.

FIGS. 6A-6B are graphs showing that vaccinating against Pel induces high anti-Pel antibody titers. Total IgG antibody titer from 14 days post-primary vaccination (FIG. 6A) and post-secondary vaccination (FIG. 6B) for Pel antigen are shown with and without the adjuvants Alhydrogel and INI-2002. Mock-immunized mice were injected with 2% glycerol. Asterisks denote statistical significance versus the mock vaccination controls (2% glycerol). Ordinary one-way ANOVA followed by Fisher's LSD were used to determine statistical analysis where *=p≤0.05, **=p≤0.01, and ***=p≤0.001.

### DETAILED DESCRIPTION

Section headings as used in this section and the entire disclosure herein are merely for organizational purposes and are not intended to be limiting.

### 1. Definitions

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

As used herein, “isolated” refers to a polynucleotide, polypeptide or other component that is removed from at least one component with which it is naturally associated. For example, an isolated polynucleotide, polypeptide or other component may be present outside the cell in which it is typically found in nature, whether purified or not. Additionally, or alternatively, the isolated component is found in a context other than that in which it is naturally found, e.g., separated from nucleotide sequences with which it typically is in proximity in nature, or adjacent (or contiguous with) nucleotide sequences with which it typically is not in proximity. Optionally, an isolated component, e.g., polynucleotide or polypeptide, may be subjected to one or more enrichment or purification procedures, e.g., cell lysis, extraction, centrifugation, precipitation, or the like.

As used herein, the term “preventing” refers to partially or completely delaying onset of an infection, disease, disorder and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

As used herein, “treat,” “treating” and the like means a slowing, stopping or reversing of progression of a disease or disorder when provided a composition described herein to an appropriate control subject. The term also means a reversing of the progression of such a disease or disorder to a point of eliminating or greatly reducing the cell proliferation. As such, “treating” means an application or administration of the compositions described herein to a subject, where the subject has a disease or a symptom of a disease, where the purpose is to cure, heal, alleviate, relieve, alter, remedy, ameliorate, improve or affect the disease or symptoms of the disease.

A “subject” or “patient” may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods and compositions provided herein, the mammal is a human.

As used herein, the terms “providing”, “administering,” “introducing,” are used interchangeably herein and refer to the placement of the compositions of the disclosure into a subject by a method or route which results in at least partial localization of the composition to a desired site. The compositions can be administered by any appropriate route which results in delivery to a desired location in the subject.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### 2. Isolated Polysaccharide Compositions

Disclosed herein are compositions comprising an isolated polysaccharide comprising β-1,4 linked galactosamine and glucosamine monomers, wherein the amino groups of each of the galactosamine and glucosamine are partially substituted with acetate.

In some embodiments, less than 100% of glucosamine and galactosamine amino groups are substituted with acetate. In certain embodiments, less than 99%, less than 95%, less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10⁰%6, less than 5%, or less than 1% of glucosamine and galactosamine amino groups are substituted with acetate.

In some embodiments, 0-99% of glucosamine and galactosamine amino groups are substituted with acetate. In certain embodiments, 1-99%, 5-99%, 10-99%, 15-99%, 20-99%, 25-99%, 30-99%, 35-99%, 40-99%, 45-99%, 50-99%, 55-99%, 60-99%, 65-99%, 70-99%, 75-99%, 80-99%, 85-99%, 90-99%, 95-99%, 1-95%, 5-95%, 10-95%, 15-95%, 20-95%, 25-95%, 30-95%, 35-95%, 40-95%, 45-95%, 50-95%, 55-95%, 60-95%, 65-95%, 70-95%, 75-95%, 80-95%, 85-95%, 90-95%, 1-90%, 5-90%, 10-90%, 15-90%, 20-90%, 25-90%, 30-90%, 35-90%, 40-90%, 45-90%, 50-90%, 55-90%, 60-90%, 65-90%, 70-90%, 75-90%, 80-90%, 85-90%, 1-85%, 5-85%, 10-85%, 15-85%, 20-85%, 25-85%, 30-85%, 35-85%, 40-85%, 45-85%, 50-85%, 55-85%, 60-85%, 65-85%, 70-85%, 75-85%, 80-85%, 1-80%, 5-80%, 10-80%, 15-80%, 20-80%, 25-80%, 30-80%, 35-80%, 40-80%, 45-80%, 50-80%, 55-80%, 60-80%, 65-80%, 70-80%, 75-80%, 1-75%, 5-75%, 10-75%, 15-75%, 20-75%, 25-75%, 30-75%, 35-75%, 40-75%, 45-75%, 50-75%, 55-75%, 60-75%, 65-75%, 70-75%, 1-70%, 5-70%, 10-70%, 15-70%, 20-70%, 25-70%, 30-70%, 35-70%, 40-70%, 45-70%, 50-70%, 55-70%, 60-70%, 65-70%, 1-65%, 5-65%, 10-65%, 15-65%, 20-65%, 25-65%, 30-65%, 35-65%, 40-65%, 45-65%, 50-65%, 55-65%, 60-65%, 1-60%, 5-60%, 10-60%, 15-60%, 20-60%, 25-60%, 30-60%, 35-0%, 40-60%, 45-60%, 50-60%, 55-60%, 1-55%, 5-55%, 10-55%, 15-55%, 20-55%, 25-55%, 30-55%, 35-55%, 40-55%, 45-55%, 50-65%, 1-50%, 5-50%, 10-50%, 15-50%, 20-50%, 25-50%, 30-50%, 35-50%, 40-50%, 45-50%, 0-45%, 1-45%, 5-45%, 10-45%, 15-45%, 20-45%, 25-45%, 30-45%, 35-45%, 40-45%, 0-40%, 1-40%, 5-40%, 10-40%, 15-40%, 20-40%, 25-40%, 30-40%, 35-40%, 0-35%, 1-35%, 5-35%, 10-35%, 15-35%, 20-35%, 25-35%, 30-35%, 0-30%, 1-30%, 5-30%, 10-30%, 15-30%, 20-30%, 25-30%, 0-25%, 1-25%, 5-25%, 10-25%, 15-25%, 20-25%, 0-20%, 1-20%, 5-20%, 10-20%, 15-20%, 0-15%, 1-15%, 5-15%, 10-15%, 0-10%, 1-10%, 5-10%, 0-55%, 1-5%, or 0-1% of glucosamine and galactosamine amino groups are substituted with acetate. In some embodiments, none of the glucosamine and galactosamine amino groups are substituted with acetate.

In particular embodiments, 1-99/a, 5-95%, 10-90%, 15-85%, 20-80%, 25-75%, 30-70%, 35-65%, 40-60%, or 0-1% of glucosamine and galactosamine amino groups are substituted with acetate. In other embodiments, 1-99% of glucosamine and galactosamine amino groups are substituted with acetate. In still other embodiments, 5-95% of glucosamine and galactosamine amino groups are substituted with acetate. In yet other embodiments, 10-90% of glucosamine and galactosamine amino groups are substituted with acetate. In still other embodiments, 15-85% of glucosamine and galactosamine amino groups are substituted with acetate. In still yet other aspects, 20-80% of glucosamine and galactosamine amino groups are substituted with acetate. In still further aspects, 25-75% of glucosamine and galactosamine amino groups are substituted with acetate. In still further aspects, 30-70% of glucosamine and galactosamine amino groups are substituted with acetate. In yet further aspects, 35-65% of glucosamine and galactosamine amino groups are substituted with acetate. In still yet further aspects, 40-60% of glucosamine and galactosamine amino groups are substituted with acetate. In still yet further aspects, 0-1% of glucosamine and galactosamine amino groups are substituted with acetate.

In select embodiments, less than 60% of glucosamine and galactosamine amino groups are substituted with acetate. In certain embodiments, less than 50%, less than 55%, less than 45%, less than 40%, less than 35%, less than 30%, less than 25%, less than 20%, less than 15%, less than 10⁰%, less than 5%, or less than 1% of glucosamine and galactosamine amino groups are substituted with acetate. In some embodiments, 0-60%, 1-55%, 5-50%, or 10-45% of glucosamine and galactosamine amino groups are substituted with acetate. In yet other embodiments, 0-60% of glucosamine and galactosamine amino groups are substituted with acetate. In still yet other embodiments, 1-55% of glucosamine and galactosamine amino groups are substituted with acetate. In still further embodiments, 5-50% of glucosamine and galactosamine amino groups are substituted with acetate. In still further embodiments, 10-45% of glucosamine and galactosamine amino groups are substituted with acetate.

In some embodiments, the composition is sterile.

In some embodiments, the composition comprises greater than 70% of the isolated polysaccharide. In some embodiments, the composition comprises greater than 75%, greater than 80%, greater than 85%, greater than 90%, greater than 95%, greater than 96%, greater than 97%, greater than 98%, or greater than 99% of the isolated polysaccharide.

In some embodiments, the isolated polysaccharide comprises the structure:

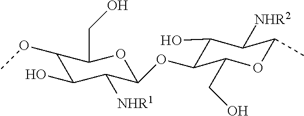

wherein R¹ and R² are independently selected from hydrogen and C(O)CH₃.

In some embodiments, less than 100% of R¹ and R² are C(O)CH₃. In some embodiments, less than 99%, less than 95%, less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less than 5%, or less than 1% of R¹ and R² are C(O)CH₃.

In some embodiments, 0-99% of R¹ and R² are C(O)CH₃. In certain embodiments, 1-99%, 5-99%, 10-99%, 15-99%, 20-99%, 25-99%, 30-99%, 35-99%, 40-99%, 45-99%, 50-99%, 55-99%, 60-99%, 65-99%, 70-99%, 75-99%, 80-99%, 85-99%, 90-99%, 95-99%, 1-95%, 5-95%, 10-95%, 15-95%, 20-95%, 25-95%, 30-95%, 35-95%, 40-95%, 45-95%, 50-95%, 55-95%, 60-95%, 65-95%, 70-95%, 75-95%, 80-95%, 85-95%, 90-95%, 1-90%, 5-90%, 10-90%, 15-90%, 20-90%, 25-90%, 30-90%, 35-90%, 40-90%, 45-90%, 50-90%, 55-90%, 60-90%, 65-90%, 70-90%, 75-90%, 80-90%, 85-90%, 1-85%, 5-85%, 10-85%, 15-85%, 20-85%, 25-85%, 30-85%, 35-85%, 40-85%, 45-85%, 50-85%, 55-85%, 60-85%, 65-85%, 70-85%, 75-85%, 80-85%, 1-80%, 5-80%, 10-80%, 15-80%, 20-80%, 25-80%, 30-80%, 35-80%, 40-80%, 45-80%, 50-80%, 55-80%, 60-80%, 65-80%, 70-80%, 75-80%, 1-75%, 5-75%, 10-75%, 15-75%, 20-75%, 25-75%, 30-75%, 35-75%, 40-75%, 45-75%, 50-75%, 55-75%, 60-75%, 65-75%, 70-75%, 1-70%, 5-70%, 10-70%, 15-70%, 20-70%, 25-70%, 30-70%, 35-70%, 40-70%, 45-70%, 50-70%, 55-70%, 60-70%, 65-70%, 1-65%, 5-65%, 10-65%, 15-65%, 20-65%, 25-65%, 30-65%, 35-65%, 40-65%, 45-65%, 50-65%, 55-65%, 60-65%, 1-60%, 5-60%, 10-60%, 15-60%, 20-60%, 25-60%, 30-60%, 35-0%, 40-60%, 45-60%, 50-60%, 55-60%, 1-55%, 5-55%, 10-55%, 15-55%, 20-55%, 25-55%, 30-55%, 35-55%, 40-55%, 45-55%, 50-65%, 1-50%, 5-50%, 10-50%, 15-50%, 20-50%, 25-50%, 30-50%, 35-50%, 40-50%, 45-50%, 0-45%, 1-45%, 5-45%, 10-45%, 15-45%, 20-45%, 25-45%, 30-45%, 35-45%, 40-45%, 0-40%, 1-40%, 5-40%, 10-40%, 15-40%, 20-40%, 25-40%, 30-40%, 35-40%, 0-35%, 1-35%, 5-35%, 10-35%, 15-35%, 20-35%, 25-35%, 30-35%, 0-30%, 1-30%, 5-30%, 10-30%, 15-30%, 20-30%, 25-30%, 0-25%, 1-25%, 5-25%, 10-25%, 15-25%, 20-25%, 0-20%, 1-20%, 5-20%, 10-20%, 15-20%, 0-15%, 1-15%, 5-15%, 10-15%, 0-10%, 1-10%, 5-10%, 0-55%, 1-5%, or 0-1% of R¹ and R² are C(O)CH₃. In some embodiments, R¹ and R² at every occurrence are hydrogen.

In particular embodiments, 1-99%, 5-95%, 10-90%, 15-85%, 20-80%, 25-75%, 30-70%, 35-65%, 40-60%, or 0-1% of R¹ and R² are C(O)CH₃. In some other embodiments 1-99% of R¹ and R² are C(O)CH₃. In yet other embodiments, 5-95% of R¹ and R² are C(O)CH₃. In still yet other embodiments, 10-90% of R¹ and R² are C(O)CH₃. In yet further embodiments, 15-85% of R¹ and R² are C(O)CH₃. In yet further embodiments, 20-80% of R¹ and R² are C(O)CH₃. In still further embodiments, 20-80% of R¹ and R² are C(O)CH₃. In yet still further embodiments, 25-75% of R¹ and R² are C(O)CH₃. In yet further embodiments, 30-70% of R¹ and R² are C(O)CH₃. In still further embodiments, 35-65% of R¹ and R² are C(O)CH₃. In yet still further embodiments, 40-60% of R¹ and R² are C(O)CH₃. In still further embodiments, 0-1% of R¹ and R² are C(O)CH₃.

In select embodiments, less than 60% of R¹ and R² are C(O)CH₃. In certain embodiments, less than 50%, less than 55%, less than 45%, less than 40%, less than 35%, less than 30%, less than 25%, less than 20%, less than 15%, less than 10%, less than 5%, or less than 1% of R¹ and R² are C(O)CH₃. In some embodiments, 0-60%, 1-55%, 5-50%, or 10-45% of R¹ and R² are C(O)CH₃. In yet other embodiments, 0-60% of R¹ and R² are C(O)CH₃. In still yet other embodiments, 1-55% of R¹ and R² are C(O)CH₃. In still further embodiments, 5-50% of R¹ and R² are C(O)CH₃. In still further embodiments, 10-45% of R¹ and R² are C(O)CH₃.

In some embodiments, the isolated polysaccharide is conjugated to a carrier. In some embodiments, the carrier is a peptide carrier. In select embodiments, the carrier is a cross-reactive material (CRM) or tetanus toxin (TT).

In some embodiments, the isolated polysaccharide is conjugated to the carrier with a linker.

### 3. Pseudomonas Vaccine

P. aeruginosa bacterial infections are a major public health threat P. aeruginosa is an opportunistic bacterial pathogen that causes severe and life-threatening infections especially in the critically ill, immunocompromised patients, and patients with the genetic disorder cystic fibrosis. The incidence and economic burden of P. aeruginosa infections is high. P. aeruginosa causes an estimated 10% of all hospital-acquired infections, which affect 1.7 million patients and cost $5 billion in medical care annually in the US alone. P. aeruginosa is present in 5-10/a of chronically infected wounds, which affect 6.5 million patients in the US and cost an estimated $25 billion annually. P. aeruginosa is the primary respiratory pathogen associated with the genetic disease cystic fibrosis which affects one in every 2000 newborns and costs an estimated $310,000 per year per patient for the latest FDA-approved treatment.

Antibiotics are not effective against chronic P. aeruginosa infections. While antibiotic treatment has improved the management of P. aeruginosa infections, once a P. aeruginosa infection is established, it can be incredibly difficult to eradicate. A prime example of this are P. aeruginosa infections in the cystic fibrosis lung which are associated with persistent inflammation, accelerated lung disease, and mortality. Despite repeated courses of antibiotics, P. aeruginosa infections persist in the cystic fibrosis airways and are rarely if ever cleared. Moreover, multidrug resistant bacteria contribute to a serious restriction in treatment options for P. aeruginosa infections. Frequently strains of P. aeruginosa are becoming resistant to the best available and last-resort antibiotics. Because of this, the World Health Organization has identified P. aeruginosa as a priority pathogen of the greatest threat to human health. Alternative treatment options for this deadly pathogen are urgently needed.

The Pel polysaccharide promotes chronic P. aeruginosa infections. Part of what makes P. aeruginosa infections so difficult to clear is that P. aeruginosa cells rarely grow alone. In many laboratory and infectious environments, P. aeruginosa grows as a biofilm or aggregate of bacterial cells embedded in a polymer rich matrix. Extracellular polysaccharides are important components of the biofilm matrix that contribute to the persistence of biofilm infections by promoting resistance to antimicrobials and the immune system.

A P. aeruginosa vaccine could prevent life-threatening infections. Individuals with high risk factors for P. aeruginosa infections including patients with extended hospital stays, and patients with diabetes or cystic fibrosis are prime candidates for vaccination. Thus, vaccinating individuals at high risk for P. aeruginosa infections would thereby eliminate the suffering of many.

It is well established that the lipopolysaccharide (LPS) 0-antigen of P. aeruginosa elicits a protective immune response in animals. However, broad-based protection against the 20 different serotypes of P. aeruginosa LPS would require the development of complex and costly multivalent vaccines. Alternatively, the lipid A of P. aeruginosa LPS is a much more highly conserved target, but toxicity issues prevent further development as a target antigen. Further hindering the clinical development of LPS-targeted vaccines is the ability of P. aeruginosa to evade host defenses by modifying its LPS during infection.

A Pel or Pel-conjugate vaccine may be broadly protective against diverse bacterial infections since the core Pel operon is present in a variety of bacterial pathogens including Bacilli, Clostridia. Streptococci, and Actinobacteria.

Disclosed herein is an anti-Pseudomonas vaccine that targets the Pel polysaccharide to prevent P. aeruginosa infections. The vaccine comprises the isolated polysaccharide compositions described herein in Section 2.

The vaccine may comprise an adjuvant or immunostimulant. Adjuvants and immunostimulants are compounds that either directly or indirectly stimulate the immune system's response to a co-administered antigen. Suitable adjuvants are commercially available as, for example, Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham); mineral salts (for example, aluminum, silica, kaolin, and carbon); aluminum salts such as aluminum hydroxide gel (alum), AlK(SO₄)₂, AlNa(SO₄)₂, AlNH₄(SO₄), and Al(OH)₃; salts of calcium (e.g., Ca₃(PO₄)₂), iron or zinc; an insoluble suspension of acylated tyrosine; acylated sugars; cationically or anionically derivatized polysaccharides; polynucleotides (for example, poly IC and poly AU acids); polyphosphazenes; cyanoacrylates; polymerase-(DL-lactide-co-glycoside); biodegradable microspheres; liposomes; lipid A and its derivatives; monophosphoryl lipid A; wax D from Mycobacterium tuberculosis, as well as substances found in Corynebacterium parvum, Bordetella pertussis, and members of the genus Brucella); bovine serum albumin; diphtheria toxoid; tetanus toxoid; edestin; keyhole-limpet hemocyanin; Pseudomonal Toxin A; choleragenoid; cholera toxin; pertussis toxin; viral proteins; and Quil A. Aminoalkyl glucosamine phosphate compounds can also be used (see, e.g., WO 98/50399, U.S. Pat. No. 6,113,918 (which issued from U.S. Ser. No. 08/853,826), and U.S. Ser. No. 09/074,720, incorporated herein by reference). The adjuvants may drive a Th1, Th2, or Th17 response. The adjuvants may be those described in WO2019169313, WO2019165114, and WO2019197595, incorporated herein by reference.

In addition, adjuvants such as cytokines (e.g., GM-CSF or interleukin-2, -7, or -12), interferons, or tumor necrosis factor, may also be used as adjuvants. Protein and polypeptide adjuvants may be obtained from natural or recombinant sources according to methods well known to those skilled in the art. When obtained from recombinant sources, the adjuvant may comprise a protein fragment comprising at least the immunostimulatory portion of the molecule. Other known immunostimulatory macromolecules which can be used include, but are not limited to, polysaccharides, tRNA, non-metabolizable synthetic polymers such as polyvinylamine, polymethacrylic acid, polyvinylpyrrolidone, mixed polycondensates (with relatively high molecular weight) of 4′,4-diaminodiphenylmethane-3,3′-dicarboxylic acid and 4-nitro-2- aminobenzoic acid (See, Sela, M., Science 166: 1365-1374 (1969)) or glycolipids, lipids or carbohydrates.

Vaccine preparation is a well-developed art and general guidance in the preparation and formulation of vaccines is readily available from any of a variety of sources. One such example is New Trends and Developments in Vaccines, edited by Voller et al., University Park Press, Baltimore, Md., U.S.A. 1978.

The vaccines of the present disclosure may also contain other compounds, which may be biologically active or inactive. For example, one or more immunogenic portions of other antigens may be present, either incorporated into a fusion polypeptide or as a separate compound, within the vaccine. The vaccines may generally be used for prophylactic and therapeutic purposes.

The vaccines may be formulated for any appropriate manner of administration, and thus administered, including for example, topical, oral, nasal, intravenous, intravaginal, epicutaneous, sublingual, intracranial, intradermal, intraperitoneal, subcutaneous, intramuscular administration, or via inhalation.

The vaccines may also comprise buffers (e.g., neutral buffered saline or phosphate buffered saline), carbohydrates (e.g., glucose, mannose, sucrose or dextrans), mannitol, proteins, polypeptides or amino acids such as glycine, antioxidants, bacteriostats, chelating agents such as EDTA or glutathione, solutes that render the formulation isotonic, hypotonic or weakly hypertonic with the blood of a recipient, suspending agents, thickening agents and/or preservatives. Alternatively, vaccines may be formulated as a lyophilisate. Compounds may also be encapsulated within liposomes using technology and techniques well known to those skilled in the art.

### 4. Methods of Use

The present disclosure provides methods for treating, reducing or preventing Pseudomonas aeruginosa infection in a subject in need thereof. The methods include administering to the subject an effective amount of the compositions or vaccines disclosed herein.

The present disclosure provides methods for treating, reducing or preventing Pseudomonas aeruginosa lung infection in a subject suffering from cystic fibrosis. The methods include administering to the subject an effective amount of the compositions or vaccines disclosed herein.

An “effective amount” of the compositions or vaccines disclosed herein is an amount that is delivered to a subject, either in a single dose or as part of a series, which is effective for inducing an immune response against Pseudomonas aeruginosa in the subject. This amount varies depending upon the health and physical condition of the subject to be treated, the capacity of the subject's immune system to synthesize antibodies, the formulation of the peptides, compositions or vaccine, and other relevant factors. The amount of polysaccharide in each vaccine dose is generally selected as an amount which induces an immunoprotective response without significant adverse side effects as seen in other vaccines. Of course, the dosage administered may be dependent upon the age, weight, kind of concurrent treatment, if any, and nature of the polysaccharide administered. It is expected that the amount will fall in a relatively broad range that can be determined by one of skill in the art through routine trials.

For example, an effective amount of a compound of the compositions described here, may contain about 0.0001 mg to about 1 mg, about 0.01 mg to about 1 mg, about 0.1 mg to about 1 mg, about 0.0001 mg to about 0.9 mg, about 0.01 mg to about 0.9 mg, about 0.1 mg to about 0.9 mg, about 0.0001 mg to about 0.8 mg, about 0.01 mg to about 0.8 mg, about 0.1 mg to about 0.8 mg, about 0.0001 mg to about 0.7 mg, about 0.01 mg to about 0.7 mg, about 0.1 mg to about 0.7 mg, about 0.0001 mg to about 0.6 mg, about 0.01 mg to about 0.6 mg, about 0.1 mg to about 0.6 mg, about 0.0001 mg to about 0.5 mg, about 0.01 mg to about 0.5 mg, about 0.1 mg to about 0.5 mg, about 0.0001 mg to about 0.4 mg, about 0.01 mg to about 0.4 mg, about 0.1 mg to about 0.4 mg, about 0.0001 mg to about 0.3 mg, about 0.01 mg to about 0.3 mg, about 0.1 mg to about 0.3 mg, about 0.0001 mg to about 0.2 mg, about 0.01 mg to about 0.2 mg, about 0.1 mg to about 0.2 mg, about 0.0001 mg to about 0.1 mg, about 0.01 mg to about 0.1 mg, about 0.0001 mg to about 0.01 mg, or about 0.0001 mg to about 0.001 mg of the Pel polysaccharide.

The compositions of vaccines disclosed herein can be administered in a wide variety of therapeutic dosage forms in the conventional vehicles for topical, oral, systemic, local, and parenteral administration.

The route and regimen of administration will vary depending upon the population and the indication for vaccination and is to be determined by the skilled practitioner. For example, the vaccines disclosed herein may be administered in such oral dosage forms for example as tablets, capsules (each including timed release and sustained release formulations), pills, powders, granules, elixirs, tinctures, solutions, suspensions, syrups and emulsions, or by injection. Similarly, they may also be administered parentally, e.g., in intravenous (either by bolus or infusion methods), intraperitoneal, subcutaneous, topical with or without occlusion, or intramuscular form.

The administration may comprise an initial immunization or dose and at least one subsequent immunization or booster dose, following known standard immunization protocols. The boosting doses will be adequately spaced at such times where the levels of circulating antibody fall below a desired level. Boosting doses may consist of one or both of the peptides disclosed herein and may comprise alternative carriers and/or adjuvants. The booster dosage levels may be the same or different that those of the initial immunization dosage.

The specific dose level may depend upon a variety of factors including the activity of the peptide, composition or vaccine, the age, body weight, general health, and diet of the subject, time of administration, and route of administration.

The compositions and vaccines may be prepared, packaged, or sold in a form suitable for bolus administration or sold in unit dosage forms, such as in ampules or multi-dose containers containing a preservative.

The present disclosure provides for the use of the compositions disclosed herein in the manufacture of a medicament for the treatment or prevention of Pseudomonas aeruginosa infections.

### 5. Examples

### Example 1

### Purification of Pel Polysaccharide from P. aeruginosa

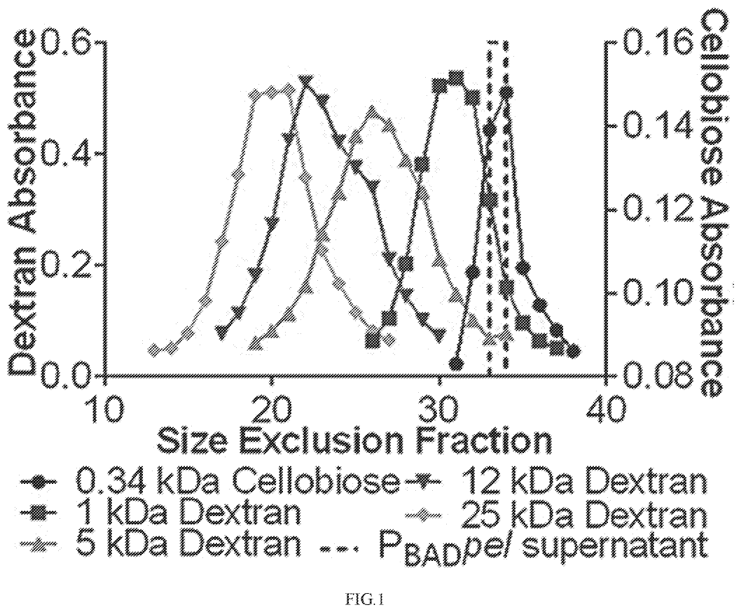

Size exclusion chromatography indicates that Pel has two forms, a larger cell-associated form which is greater than 80 kDa and a smaller secreted form that is 0.5 kDa (FIG. 1). Secreted Pel can be purified from an overproduction strain in sufficient quantity, purity, stability for the testing.

Strains were cultivated on Jensen's medium which contained NaCl (85.6 mM), K₂HPO₄ (14.4 mM), sodium glutamate (92 mM), valine (24 mM), phenylalanine (8 mM), glucose (70 mM), MgSO₄ (1.33 mM), CaCl₂ (0.14 mM), FeSO₄ (0.0039 mM), and ZnSO₄ (0.0085 mM) at pH 7.3.

Pel polysaccharides were purified by harvesting the supernatant from a strain engineered to overproduce Pel in the presence of arabinose PAO1 ΔwspF Δpsl P_{BAD}pel (herein P_{BAD}pel). Jensen's medium (50 mL) was inoculated with 0.5 mL of overnight culture of P_{BAD}pel. Cultures were grown for 20 h at 37° C. with constant shaking. To obtain secreted Pel, the supernatant was harvested by centrifugation (8,300×g for 15 min at 22° C.). Secreted Pel was precipitated for 1 h at 4° C. with ethanol (final concentration 75% vol/vol). The precipitate was washed three times with 95-100% (vol/vol) ethanol, resuspended in 2 mL of buffer (1 mM CaCl₂) and 2 mM MgCl₂ in 50 mM Tris, pH 7.5), and treated with 5 mg DNase I and 5 mg RNase A for 2 h at 37° C., followed by 5 mg of proteinase K overnight at 37° C. The precipitated polysaccharides were dialyzed against water (100 kDa molecular-weight cut off), followed by mild sonication and treatment with polymyxin B to remove endotoxins.

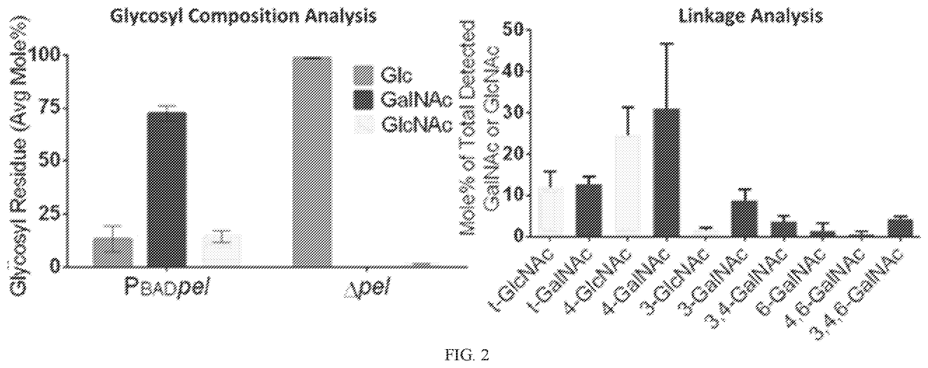

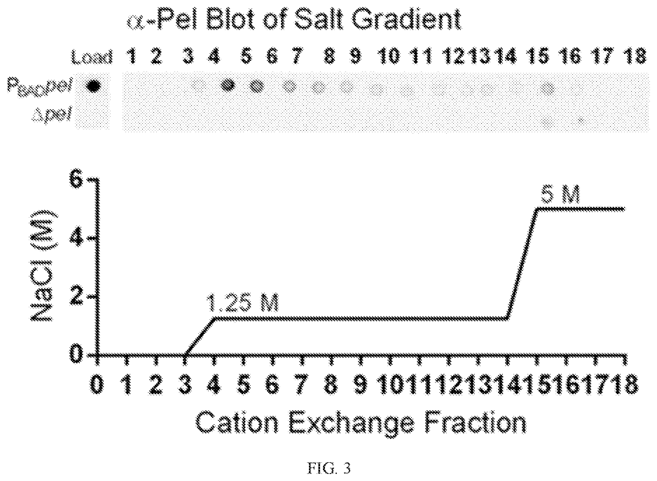

The Pel polysaccharide, which is encoded by a seven-gene operon (pelA-pelG), is an extracellular polysaccharide produced by P. aeruginosa. Pel promotes chronic P. aeruginosa infections by enhancing the structural integrity of biofilms and increasing the tolerance of the bacteria to antibiotics. Although previous reports suggested that glucose was a primary component of Pel, it was found that Pel is a cationic amino sugar polymer composed of partially acetylated β-1,4 glycosidic linkages of N-acetylgalactosamine (GalNAc) and N-acetylglucosamine (GlcNAc) (FIG. 2). Glycosyl composition analysis indicates that Pel is rich in N-acetylgalactosamine (GalNAc; 72.5±3.6 mol %) and N-acetylglucosamine (GlcNAc; 14.5 t 2.6 mol %). In controls lacking pel genes, GalNAc was below detection, and GlcNAc was 1.4±0.3 mol %. The ratio of GalNAc to GlcNAc was 5:1 (0.7) and could indicate either a minimum repeating unit or a random incorporation of GlcNAc into the polysaccharide. Pel, detected with anti-Pel immunoblot, bound a strong cation exchange column indicating that the polysaccharide has an overall positive charge and that the GalNAc and GlcNAc sugars are partially deacetylated (FIG. 3).

Polyclonal antibodies raised against purified Pel in rabbits indicated that Pel is immunogenic (FIG. 3 top).

### Example 2

### Development of Anti-Pel Vaccine

The Pel polysaccharide will be purified as described above. The Limulus Amebocyte Lysate (LAL) test will be used to confirm that the purified Pel is endotoxin free. If required, Pel can be further purified by size exclusion. The Pel polysaccharide may be coupled to a carrier protein such as cross-reactive materials (CRM) or tetanus toxin (TT) to induce a stronger immune response.

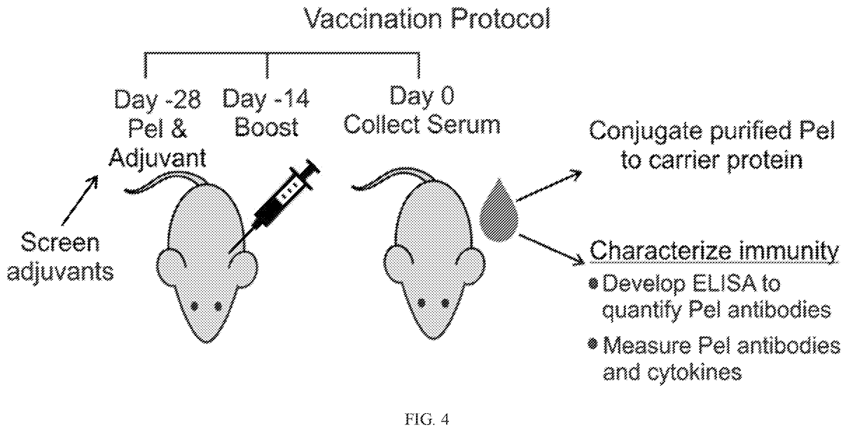

A quantitative ELISA will be developed for detecting serum IgG, IgG1 and IgG2 polyclonal antibodies against Pel. For these studies, serum collected from mice immunized with the Pel polysaccharide conjugate will be used. Mice (C57B16, n=30, 50% male/female) will be vaccinated twice via intramuscular injection with 14 days between vaccinations (FIG. 4). Serum from mock-immunized mice (PBS) will serve as an additional control to establish sensitivity and lower limit of quantitation for the assay. Purified Pel will be the target antigen for the ELISA assays. Control antigens will include plates coated with Psl, a different extracellular polysaccharide produced by P. aeruginosa.

Initially, the magnitude and duration of anti-Pel vaccine responses in 8-10 C57B16 mice per group immunized with the Pel conjugate will be defined without an adjuvant. Mice will be vaccinated twice via intramuscular injection with 14 days between the primary and secondary vaccinations (FIG. 4). Cell-mediated immunity will be evaluated in a subset of 4 mice per group by measuring Th1/Th2/Th17 cytokine levels in splenocyte cultures (assayed by multiplex MSD assay) and/or ELISPOT assay to determine the frequency of antigen specific CD4 and CD8 T-cells. Humoral immunity will be measured in 8-10 mice/group via assessments of Pel-specific serum IgG1 and IgG2b at 14 days following each vaccination. The magnitude of these antibody responses will be measured using the ELISA assays as described above. Secondary endpoints will include assessment of vaccine safety through weight gain/loss and monitoring of observable clinical symptoms (ruffled fur, hunched posture) following vaccination.

Having defined baseline responses to immunization against the Pel conjugate without an adjuvant, the immunogenicity of the Pel conjugate with adjuvants driving either Th1, Th2, or Th17 responses will be evaluated. The endpoints will be readouts of cellular and humoral immunity, as described above.

### Example 3

### Pel Immunity

Vaccinating against amino sugar polysaccharides is effective at preventing S. aureus and E. coli bacterial infections. However, no vaccine is available for the prevention of P. aeruginosa infections in humans. Mice will be vaccinated against Pel and infection pathogenesis will be monitored in a mouse model of pneumonia.

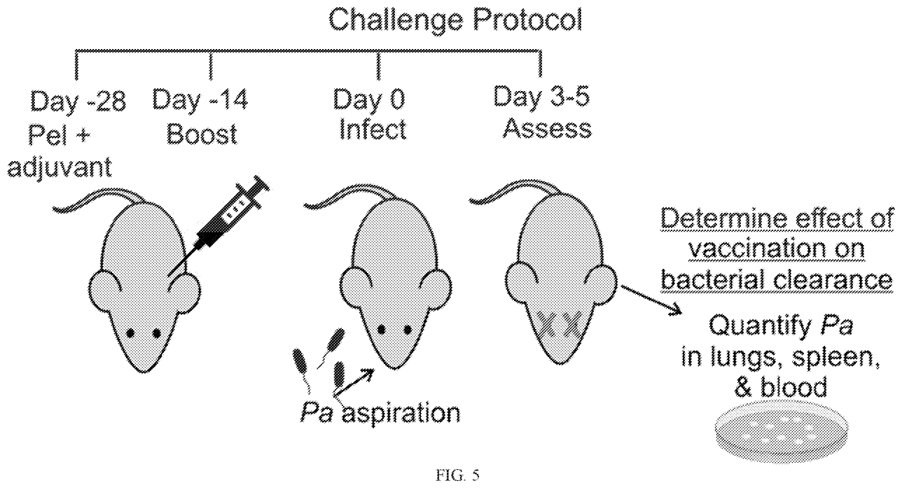

The ability of anti-Pel immunity to prevent P. aeruginosa infections will be tested in a murine pneumonia challenge model (FIG. 5). Twenty-eight days prior to infection, mice (C57B16, 8 mice for each condition) will be vaccinated with the Pel-conjugate vaccine, with or without an adjuvate, or a PBS mock vaccine, followed by a booster vaccine 14 days later. The mice will be challenged with P. aeruginosa strain PAO1 that produces Pel or a pel-deletion mutant. Control mice will be challenged with sterile PBS. Bacteria will be introduced to the mice lung by oropharyngeal aspiration. Inoculating doses will be determined empirically. Viable bacteria will be enumerated in homogenized lungs, spleens, and blood in immunized and mock-immunized animals at 3, 4, and 5 days post-infection.

### Example 4

### Immune Activation by Pe Polysaccharide

The Pel polysaccharide was purified by harvesting the supernatant from a strain engineered to overproduce Pel in the presence of 0.5% arabinose PAO1 ΔwspF Δpsl ΔR2 ΔwbpL P_{BAD}pel (herein P_{BAD}pel). To reduce endotoxin contamination in polysaccharide preparations, the overexpression strain included a wbpL deletion, which rendered it deficient in A-Band and B-Band lipopolysaccharides. P_{BAD}pel was cultivated on Jensen's defined medium which contained NaCl (85.6 mM), K₂HPO₄ (14.4 mM), sodium glutamate (92 mM), valine (24 mM), phenylalanine (8 mM), glucose (70 mM), MgSO₄ (1.33 mM), CaCl₂ (0.14 mM), FeSO₄ (0.0039 mM), and ZnSO₄ (0.0085 mM) at pH 7.3.

Jensen's medium (100 mL) was inoculated with 100 μl of overnight culture of P_{BAD}pel. Cultures were grown for 18 h at 37° C. with constant shaking. To obtain secreted Pel, the supernatant was harvested by centrifugation (8,300×g for 15 min at 22° C.). Secreted Pel was precipitated for 1 h at −20° C. with ethanol (final concentration 75% vol/vol). The precipitate was washed three times with 95-100% (vol/vol) ethanol, resuspended in 2 mL of buffer (1 mM CaCl₂) and 2 mM MgCl₂ in 50 mM Tris, pH 7.5), and treated with 5 mg DNase I and 5 mg RNase A for 2-3 h at 37° C., followed by 5 mg of proteinase K overnight at 37° C. Proteinase K-treated samples were ethanol precipitated again and washed with 95-100% Y, ethanol. The precipitated polysaccharides were dialyzed against water (100 kDa molecular-weight cut off). Dialyzed samples containing purified Pel were lyophilized and stored at 4° C. until use.

Pel was formulated by weighing 24.10 mg of the purified material and bringing it to 12 μg/ul in 2% glycerol. The mixture was divided into two 1 mL vials, each sonicated for 1 hour at 30° C. in a Covaris Focused Ultrasonicator to break up Pel aggregates before combining and sonicating for an additional 30 minutes. The final sonicated formulation was used for injections as noted below.

A quantitative ELISA was developed for detecting total serum IgG polyclonal antibodies against Pel. For these studies, serum collected from mice immunized with purified Pel polysaccharide was used. Mice (BALB/c, n=5, female) were vaccinated twice via intramuscular injection with 14 days between vaccinations. Serum from mock-immunized mice (2% glycerol) served as a negative control to establish sensitivity and lower limit of quantitation for the assay.

To determine the immunogenicity of Pel, female BALB/c mice ages 6-8 weeks old (5 mice/group) were vaccinated by intramuscular injection in the left hind limb with the purified Pel polysaccharide antigen with and without adjuvant. Pel antigen doses were 10 μg and 100 μg of purified Pel. Immunostimulatory adjuvants tested were (i) Alhydroge® (alum) at 2× the antigen concentration and (ii) Alhydrogel plus adjuvant INI-2002 (1 μg), a TLR4 agonist. Mice were vaccinated twice with 14 days between the primary and secondary vaccinations. Fourteen days after the primary injection submandibular bleeds were performed, and serum was separated using Microtainer Serum Separator Tubes (BD Biosciences) then stored at −20° C. until use. Fourteen days after the secondary injection, the mice were euthanized, and blood was collected via cardiac puncture.

Serum was analyzed via ELISA for Pel-specific total IgG antibody titers. Serum was diluted according to the expected antibody response (1:10). MaxiSorp ELISA plates (Nunc) were coated with 100 μl of 10 ug/mL Pel overnight at RT. Plates were washed 3 times with 0.05% Tween-20 in PBS (PBS-T) (Sera Care cat: 54600-0026) then blocked with EIA buffer (1% BSA, 0.1% tween-20, 5% FBS) at 37° C. for 1 h. Serum was incubated at a 1:1 ratio with lysate generated from pel-deficient P. aeruginosa strains (PAO1 ΔwspF Δpsl Δpel and PA14 ΔpelF) for 1 h at 37° C. to bind any IgG antibodies not specific for Pel. Following removal of EIA, plates were incubated with serum serial diluted in 1:10 EIA buffer (eight dilutions per sample at 1:3 dilution) for 2 h at 37° C., washed three times with PBS-T then incubated with 1:3000 anti-mouse IgG-HRP secondary antibodies in 1:10 EIA buffer for 1 h at 37° C. (Southern Biotech). Secondary antibodies were detected by addition of RT TMB Substrate (BD Biosciences) for 20 min, stopped with 50 μl 2N sulfuric acid (Ricca cat: 8310-32), followed by measurement of OD at 450 nm using a Molecular Devices SpectraMax 190 microplate reader. Serum antibody titers are reported as the dilution factor of each sample that yielded an OD of 0.300 calculated by determining the best fit line to the absorbance values of the serial dilutions and then extrapolating (XLFit, IDBS).

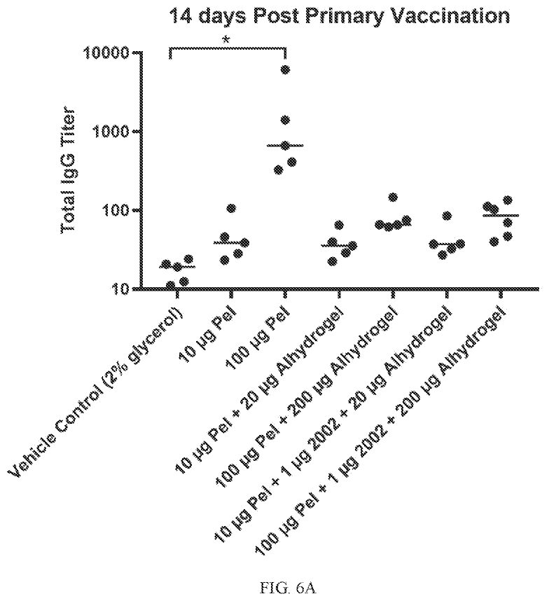

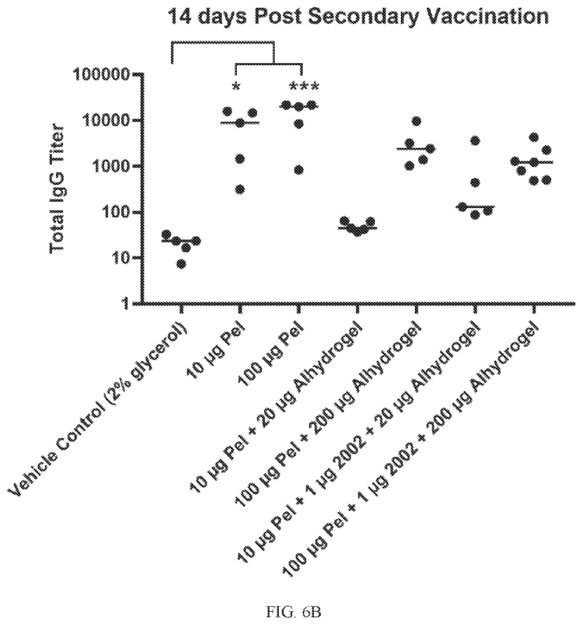

FIG. 6 shows anti-Pel serum antibody titers at 14 days post primary and 14 days post-secondary from groups vaccinated with indicated doses of Pel antigen with or without adjuvant.

Mice, except from the vehicle-control group, received either 10 μg or 100 μg of Pel antigen. The anti-Pel total IgG antibody response at 14 days post-primary increased in a Pel dose-dependent manner with the highest observed titers at doses of 100 μg Pel. At 14 days post-secondary, both doses of Pel elicited significantly higher total serum IgG than the vehicle control group. The data suggest that 100 μg of Pel is an optimal dose following a single vaccination while either 10 or 100 ug of Pel is sufficient for a prime and boost vaccine schedule.

FIG. 6 also shows the effect of immunostimulatory adjuvants on anti-Pel serum IgG. Adjuvants tested were alum alone and INI-2002 adjuvant adsorbed on alum. At the concentrations tested, both adjuvants increased the total anti-Pel serum antibody titers in relation to the vehicle control, but were generally lower than Pel-alone groups. These data suggest that Pel, in the current purified form, may not require an adjuvant to stimulate high antibody titers.

Collectively, these data demonstrate that this Pel vaccine elicits a robust immune response in mice. One or two injections induce high total anti-Pel IgG antibody titers.

For reasons of completeness, various aspects of the invention are set out in the following numbered clauses:

Clause 1. A composition comprising a polysaccharide comprising β-1,4 linked galactosamine and glucosamine monomers, wherein the amino groups of each of the galactosamine and glucosamine are partially substituted with acetate.

Clause 2. The composition of clause 1, wherein the polysaccharide is isolated.

Clause 3. The composition of clause 1 or clause 2, wherein less than 100% of the glucosamine and galactosamine amino groups are substituted with acetate

Clause 4. The composition of any of clauses 1-3, wherein 10-90% of the glucosamine and galactosamine amino groups are substituted with acetate.

Clause 5. The composition of any of clauses 1-4, wherein 20-80% of the glucosamine and galactosamine amino groups are substituted with acetate.

Clause 6. The composition of any of clauses 1-3, wherein less than 60% of glucosamine and galactosamine amino groups are substituted with acetate.

Clause 7. The composition of clause 6, wherein 1-55% of glucosamine and galactosamine amino groups are substituted with acetate.

Clause 8. The composition of clause 7, wherein 10-45% of glucosamine and galactosamine amino groups are substituted with acetate.

Clause 9. The composition of any of clauses 1-3, wherein 0-1% of glucosamine and galactosamine amino groups are substituted with acetate.

Clause 10. The composition of any of clauses 1-9, wherein the composition is sterile.

Clause 11. The composition of any of clauses 1-10 wherein the polysaccharide comprises formula (I)

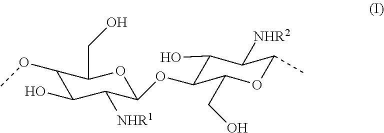

wherein R¹ and R² are independently selected from hydrogen and C(O)CH₃.

Clause 12. The composition of clause 11, wherein less than 100% of R¹ and R² are C(O)CH₃.

Clause 13. The composition of clause 11-12, wherein 10-90% of R¹ and R² are C(O)CH₃.

Clause 14. The composition of clause 11-13, wherein 20-80% of R¹ and R² are C(O)CH₃.

Clause 15. The composition of clause 11-14, wherein less than 60% of R¹ and R² are C(O)CH₃.

Clause 16. The composition of clause 15, wherein 1-55% of R¹ and R² are C(O)CH₃.

Clause 17. The composition of clause 16, wherein 10-45% of R¹ and R² are C(O)CH₃.

Clause 18. The composition of any of clauses 1-17, wherein the isolated polysaccharide is conjugated to a carrier.

Clause 19. The composition of clause 18, wherein the carrier is a peptide or protein carrier.

Clause The composition of clause 18 or clause 19, wherein the carrier is a cross-reactive material (CRM) or tetanus toxin (TT).

Clause 21. The composition of any of clauses 18-20, further comprising a linker between the isolated polysaccharide and the carrier.

Clause 22. A vaccine comprising the composition of any of clauses 1-21 and at least one adjuvant.

Clause 23. A method for treating, reducing or preventing Pseudomonas aeruginosa infection in a subject in need thereof, comprising administering to the subject an effective amount of the composition of any of clauses 1-21 or the vaccine of clause 22.

Clause 24. The method of clause 23, wherein the administering comprises an initial immunization and at least one subsequent immunization.

Clause 25. Use of the composition of any of clauses 1-21 in the manufacture of a medicament for the treatment of prevent of Pseudomonas aeruginosa infections.

Clause 26. A method for treating, reducing or preventing Pseudomonas aeruginosa lung infection in a subject suffering from cystic fibrosis, comprising administering to the subject an effective amount of the composition of any of clauses 1-21 or the vaccine of clause 22.

It is understood that the foregoing detailed description and accompanying examples are merely illustrative and are not to be taken as limitations upon the scope of the disclosure, which is defined solely by the appended claims and their equivalents.

Various changes and modifications to the disclosed embodiments will be apparent to those skilled in the art and may be made without departing from the spirit and scope thereof.

What is claimed is:

1. A composition comprising a polysaccharide comprising β-1,4 linked galactosamine and glucosamine monomers, wherein the amino groups of each of the galactosamine and glucosamine are partially substituted with acetate.

2. The composition of claim 1, wherein the polysaccharide is isolated.

3. The composition of claim 1 or claim 2, wherein less than 100% of the glucosamine and galactosamine amino groups are substituted with acetate.

4. The composition of any of claims 1-3, wherein 10-90% of the glucosamine and galactosamine amino groups are substituted with acetate.

5. The composition of any of claims 1-4, wherein 20-80% of the glucosamine and galactosamine amino groups are substituted with acetate.

6. The composition of any of claims 1-3, wherein less than 60% of glucosamine and galactosamine amino groups are substituted with acetate.

7. The composition of claim 6, wherein 1-55% of glucosamine and galactosamine amino groups are substituted with acetate.

8. The composition of claim 7, wherein 10-45% of glucosamine and galactosamine amino groups are substituted with acetate.

9. The composition of any of claims 1-3, wherein 0-1% of glucosamine and galactosamine amino groups are substituted with acetate.

10. The composition of any of claims 1-9, wherein the composition is sterile.

11. The composition of any of claims 1-10 wherein the polysaccharide comprises formula (I) 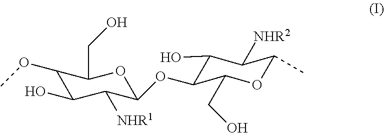
wherein R¹ and R² are independently selected from hydrogen and C(O)CH₃.

12. The composition of claim 11, wherein less than 100% of R¹ and R² are C(O)CH₃.

13. The composition of claim 11-12, wherein 10-90° %6 of R¹ and R² are C(O)CH₃.

14. The composition of claim 11-13, wherein 20-80% of R¹ and R² are C(O)CH₃.

15. The composition of claim 11-14, wherein less than 60% of R¹ and R² are C(O)CH₃.

16. The composition of claim 15, wherein 1-55% of R¹ and R² are C(O)CH₃.

17. The composition of claim 16, wherein 10-45% of R¹ and R² are C(O)CH₃.

18. The composition of any of claims 1-17, wherein the isolated polysaccharide is conjugated to a carrier.

19. The composition of claim 18, wherein the carrier is a peptide or protein carrier.

20. The composition of claim 18 or claim 19, wherein the carrier is a cross-reactive material (CRM) or tetanus toxin (TT).

21. The composition of any of claims 18-20, further comprising a linker between the isolated polysaccharide and the carrier.

22. A vaccine comprising the composition of any of claims 1-21 and at least one adjuvant.

23. A method for treating, reducing or preventing Pseudomonas aeruginosa infection in a subject in need thereof, comprising administering to the subject an effective amount of the composition of any of claims 1-21 or the vaccine of claim 22.

24. The method of claim 23, wherein the administering comprises an initial immunization and at least one subsequent immunization.

25. Use of the composition of any of claims 1-21 in the manufacture of a medicament for the treatment of prevent of Pseudomonas aeruginosa infections.

26. A method for treating, reducing or preventing Pseudomonas aeruginosa lung infection in a subject suffering from cystic fibrosis, comprising administering to the subject an effective amount of the composition of any of claims 1-21 or the vaccine of claim 22.
